(19) United States
(12) Patent Application Publication
(10) Pub. No.: US 2008/0206299 A1
(43) Pub. Date: Aug. 28, 2008

(54) Method for Recovering Minerals From Bone and Use of Same

(76) Inventor: Lawrence A. Shimp, Morganville, NJ (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/679,646

(22) Filed: Feb. 27, 2007

(51) Int. Cl. A61F 2/28

(52) U.S. Cl. 424/423

(57) ABSTRACT

Systems and methods for recovering bone minerals from bone are provided in which bone minerals are separated from bone, and the separated bone minerals with natural bone mineral inorganic moieties are isolated. Also provided are implantable hydroxyapatite materials that are at least partially derived from bone and include natural bone mineral inorganic moieties.

### FIELD OF THE INVENTION

The invention relates generally to a method for recovering minerals from bone and uses thereof. More particularly, the invention relates to bone demineralization methods that provide for the recovery of hydroxyapatite or hydroxyapatite-based products from bone.

### BACKGROUND OF THE INVENTION

Bone comprises approximately 70% mineral, i.e., hydroxyapatite (HA), 20% organic material (collagen and other proteins), and 10% water. Hydroxyapatite is a phosphate salt of calcium having the chemical formula of Ca₁₀(PO₄)₆(OH)₂. In composition it corresponds to a fully neutralized salt of phosphoric acid with additional calcium hydroxide in the crystal lattice. In bone, some calcium may be substituted with other metals, such as divalent metals including magnesium (Mg), zinc (Zn), strontium (Sr), etc. In addition, hydroxyl (OH) groups or some phosphate (PO₄) groups may be replaced with carbonate groups (CO₃).

Preserved bone growth factors found on demineralized bone are more biologically accessible when bone is demineralized, which is desirable for implantation. Accordingly, many bone demineralization processes have been developed. Bone demineralization processes that separate natural hydroxyapatite from bone are usually designed to preserve the bone organic material. Such processes typically cause the hydroxyapatite to dissolve in solution or to form other compounds. Therefore, materials used for demineralization of bone are selected so that the organic material is preserved, as opposed to preserving hydroxyapatite in its natural form, and many of the acids selected for demineralization processes cause hydroxyapatite to dissolve or react with the acid. Currently available demineralization processes leave hydroxyapatite dissolved in solution. Hydroxyapatite-derived products such as tricalcium phosphate (Ca₃(PO₄)₂, dicalcium phosphate (CaHPO₄), and natural hydroxyapatite from human bone are not recovered.

When medical implants are prepared for implantation, they may be treated with various types of materials. In certain treatments, synthetic hydroxyapatite is added to the implant, which may serve as a bone void filler or a bone grafting material. In some instances, it may be desirable to use natural hydroxyapatite, derived from bone, with its natural inorganic moieties.

In view of the aforementioned demineralization processes that treat hydroxyapatite from bone as waste, there is a need to provide a bone demineralization method in which hydroxyapatite derived from bone may be recovered.

### BRIEF SUMMARY OF THE INVENTION

A method for recovering bone minerals such as hydroxyapatite from bone is provided. More specifically, a method for recovering bone minerals such as hydroxyapatite from bone using extraction methods is provided. Recovered bone minerals may be used as a medical implant or in combination with other medical implants.

In one embodiment a method for recovering minerals from bone is provided. The method comprises separating bone minerals from bone and isolating the separated bone minerals such that the bone minerals are isolated with natural bone mineral inorganic moieties.

In another embodiment, a method for recovering minerals from bone comprises acid treating the bone in an acid solution to demineralize the bone, where such treating with acid results in bone minerals having natural bone mineral inorganic moieties, separating the bone minerals from the acid solution, and collecting the separated bone minerals.

In yet a further embodiment, an implantable hydroxyapatite material is provided. The implantable hydroxyapatite material is at least partially derived from bone and comprises natural bone mineral inorganic moieties.

Other features and advantages of the invention will become apparent from the following detailed description, taken in conjunction with the accompanying drawings, which illustrate, by way of example, various features of embodiments of the invention.

### BRIEF DESCRIPTION OF THE DRAWINGS

FIG. 1 is a flowchart of a method for recovering hydroxyapatite from bone, in accordance with one embodiment.

FIG. 2 is a flowchart of another method for recovering hydroxyapatite from bone, in accordance with one embodiment.

FIG. 3 is another flowchart of a method for recovering hydroxyapatite from bone, in accordance with one embodiment.

FIG. 4 is a flowchart of a method for recovering hydroxyapatite from bone, in accordance with one embodiment.

FIG. 5 is an illustration of bone-derived hydroxyapatite granulates, in accordance with one embodiment.

FIG. 6 is an illustration of bone-derived hydroxyapatite paste on bone, in accordance with one embodiment.

FIG. 7 is an illustration of a medical implant with deposited bone-derived hydroxyapatite on an exterior surface, in accordance with one embodiment.

### DETAILED DESCRIPTION

Methods for recovering bone minerals such as hydroxyapatite from bone are provided. More specifically, a method for recovering bone minerals such as hydroxyapatite from bone using extraction methods is provided.

In one embodiment, a demineralization process used to demineralize bone and recover hydroxyapatite is provided. Unlike conventional demineralization processes that dissolve hydroxyapatite from bone or cause reaction of hydroxyapatite in solution to form a non-hydroxyapatite by-product, the methods discussed herein provide demineralization processes that enable recovery of hydroxyapatite or other calcium-containing products from bone.

Although the embodiments provided below describe recovery of hydroxyapatite from bone, it will be understood that other calcium-containing products may also be recovered from bone. In particular, calcium phosphates recovered from bone may include aragonite, amorphous calcium phosphate, dahllite, calcite and dentin. Recovery of various calcium phosphates from bone are described in Heinz A. Lowenstam & Stephen Weiner, On Biomineralization (Oxford University Press 1989).

In various embodiments, the methods isolate hydroxyapatite having natural bone mineral inorganic moieties. Synthetic hydroxyapatite does not have such natural bone mineral inorganic moieties. Use of hydroxyapatite recovered from bone thus may provide advantages over the use of synthetic hydroxyapatite because hydroxyapatite having natural bone mineral inorganic moieties more closely corresponds to the minerals normally found in bone tissue, in terms of content, ratios, and otherwise. This may enhance natural cell mediated bone remodeling processes. Recovered natural hydroxyapatite may be used for various applications, for example, as bone void filler, a bone grafting material, or as a supplement for medical implants, such as implants for skeletal applications. Recovered hydroxyapatite from bone may be used as a medical implant or in combination with other medical implants. For example, recovered hydroxyapatite may be used as a starting material for implant materials described in U.S. Pat. Nos. 6,294,187; 5,899,939; 6,294,041 and 6,030,635, which are hereby incorporated by reference in their entireties.

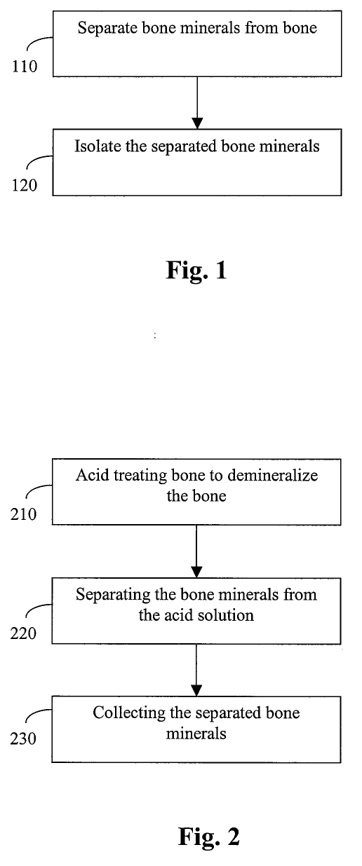

FIG. 1 is a flowchart of a method 100 for recovering bone minerals such as hydroxyapatite from bone, in accordance with one embodiment. According to FIG. 1, bone minerals are separated from bone 110, and the separated bone minerals are isolated 120. Because bone minerals naturally have inorganic moieties, when bone minerals derived from bone are separated from bone, the mineral inorganic moieties are retained in the isolated bone minerals. Further discussion of separating bone minerals from bone and isolating the separated bone minerals is provided below.

In some embodiments, a process is employed for separating natural bone minerals, such as hydroxyapatite, from bone. FIG. 2 is a flowchart of a process for recovering minerals from bone, in accordance with a further embodiment. The embodiment of FIG. 2 involves demineralizing bone, for example using acid treatment 210 of bone. Demineralizing the bone causes an acid extraction of the bone minerals from the bone. When the bone is demineralized using acid, the bone may be placed in an acid solution (or acid bath) to effect acid extraction. Bone minerals thus accumulate in the acid solution. After the bone has been demineralized, the method further comprises separating 220 the bone minerals from the acid solution and collecting 230 the separated bone minerals.

According to some embodiments, acids used for acid treating 210 in the demineralization process may include any suitable acid or mixture of acids, including but not limited to hydrochloric acid (HCl), phosphoric acid (H₃PO₄), acetic acid (C₂H₄O₂), citric acid (C₆H₈O₇), EDTA (C₁₀H₁₆N₂O₈), peracetic (C₂H₄O₃), nitric acid (HNO₃), etc. Non-oxidizing acids, which may help preserve active bone proteins, may be used. Any other suitable demineralization agents, including but not limited to calcium chelating agents at an appropriate pH, also may be used.

Separating 220 bone minerals from the acid may occur, for example, by neutralizing the acid solution containing the bone minerals with a neutralizing agent, such as a base, in order to yield a bone mineral precipitate, such as a hydroxyapatite precipitate. Where the neutralization agent is a base, any suitable base may be used for neutralization. In some embodiments, the acid and base may be selected to yield soluble salts and insoluble hydroxyapatite. For example, after acid treatment with hydrochloric acid, a sodium hydroxide (NaOH) base may be used for neutralization, reaction products of which may include soluble sodium chloride and insoluble hydroxyapatite.

In addition, or alternatively, separating 220 bone minerals from the acid may occur, for example, by neutralizing the acid and pouring off the liquid, leaving insoluble hydroxyapatite for subsequent collection. For example, separating 220 the hydroxyapatite from the acid solution may occur via neutralization to a suitable pH to cause the hydroxyapatite to precipitate out of the solution, and the collection of 230 the hydroxyapatite may occur by pouring off the liquid solution. However, it will be understood by those skilled in the art that any standard collection process may be carried out for isolation of hydroxyapatite from bone, and may include centrifugation, settling, filtration, etc.

In accordance with another embodiment, bone minerals may be recovered by demineralizing bone in an acid solution that comprises phosphoric acid, hydrochloric acid, and calcium chloride (CaCl₂), followed by neutralizing the solution with NaOH, which may yield soluble sodium chloride (NaCl), soluble excess calcium chloride, water, and insoluble hydroxyapatite. The bone demineralization process may be facilitated by increasing the temperature of the acid solution, by the use of agitation or ultrasound, by the use of pressure, or by other suitable facilitation methods.

In some embodiments, neutralization may be carried out to a pH of about 8 or above, about 9 or above, about 10 or above, about 11 or above, about 12 or above, or about 13 or above such that the neutralization pH is in the maximum stability range for hydroxyapatite. Neutralization at a higher pH may be done because at lower pH values, the hydroxyapatite may not be fully neutralized and may, for example, contain some tricalcium or dicalcium phosphate.

In further embodiments, after the solution used to demineralize the bone is neutralized, bone minerals, including hydroxyapatite, may be collected immediately. Alternatively, after the solution used to demineralize the bone is neutralized, the neutralized solution may be permitted to sit for a period of time to allow hydroxyapatite crystals to mature.

According to some embodiments, the neutralized hydroxyapatite suspension may be reduced to a neutral pH (about pH 6 to 8). Such pH reduction may be carried out by separating the insoluble hydroxyapatite and washing the hydroxyapatite. Once the hydroxyapatite is formed it may be generally insoluble (such as where the suspension pH is kept above about pH 4.5), and so the hydroxyapatite can be washed without significant loss of material. In another embodiment, a buffer may be used to reduce the pH of the hydroxyapatite suspension. Alternatively, any suitable means for reducing the hydroxyapatite suspension pH may be used. Reducing the pH of the separated hydroxyapatite to a generally neutral value may be done in order to bring the hydroxyapatite to a generally physiological pH, which may render it less irritating to tissue. In addition, a neutralization process that involves a washing step (as opposed to just a buffering step) can remove extra ions from the hydroxyapatite, which may otherwise be present and could be irritating to living tissue if implanted.

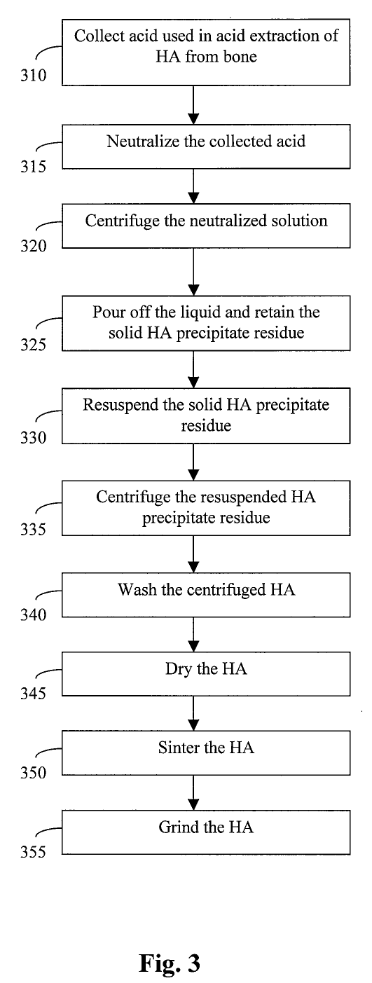

FIG. 3 illustrates a further method for recovering hydroxyapatite from bone. According to the method shown in FIG. 3, acid used in an acid extraction of hydroxyapatite from bone is collected 310. This acid may result from an acid solution used to demineralize the bone or may result from acid applied specifically to extract the hydroxyapatite without consideration of demineralization. Thus, in one embodiment of the method shown in FIG. 3, 15 cc of 0.6N HCl may be added for each gram of bone and collected after the bone has been subjected to acid demineralization for a period of 1 hour. The collected acid is neutralized 315, for example, by adding 600 cc of a 1N NaOH solution. Neutralization results in a neutralized suspension of hydroxyapatite in solution. The neutralized suspension is centrifuged 320, e.g., at a speed of 8700 rpm for 5 minutes. Liquid is poured off 325 so that water-soluble portions of the solution (derived from the bone and the base) are washed away. The solid hydroxyapatite precipitate residue can be resuspended 330, for example, in deionized water in order to further reduce the concentration of soluble substances from the bone and base. The resuspended residue is centrifuged 335, and may be resuspended again, for example in deionized water, and centrifuged (or filtered) again, so performing multiple washes 340 until the pH is reduced to a generally neutral value. The washed, solid hydroxyapatite precipitate is dried 345, for example at about 70° C. for about 16 hours, or under any other suitable drying conditions.

In accordance with any of the embodiments disclosed herein, the hydroxyapatite may be dried (for example, as described with reference to the method of FIG. 3). The dried hydroxyapatite may be further processed, for example, by sintering 350 between about 400° C. and 1250° C. for about 1 to 8 hours, then cooled, subjected to grinding 355, and sieved to a particle size of about 1 to 3 mm. It is to be recognized that other suitable processing parameters may be used, as desired. In order to confirm the composition of the recovered hydroxyapatite, an x-ray diffraction pattern may be taken of the recovered hydroxyapatite and compared to a standard pattern for hydroxyapatite.

Bone mineral recovery methods according to various embodiments may comprise chemical processes in addition to or as an alternative to those described above. Examples are described below of a variety of methods for recovering hydroxyapatite from bone, and in some embodiments, for modifying hydroxyapatite from bone.

Treatment With Mineral Acids and Other Monovalent Bases:

As discussed, separating bone minerals, such as hydroxyapatite, from bone may comprise demineralizing the bone. According to some embodiments, mineral acids other than hydrochloric acid-based solutions may be used in demineralization processes, such as nitric acid solutions. Likewise, other monovalent bases in addition to sodium hydroxide may be used in the neutralization portion of the mineral recovery process and may include, for example, potassium hydroxide. The use of monovalent bases in the neutralization portion of the hydroxyapatite recovery process may provide advantages because the amount of monovalent base added to the acid solution is not critical because any excess may be rinsed out. According to some mineral recovery processes, the acid and base may be selected such that the resulting products are insoluble hydroxyapatite and neutral salts. In addition, the neutral salts may be soluble in the recovery solution and have a lower affinity for calcium than hydroxyapatite.

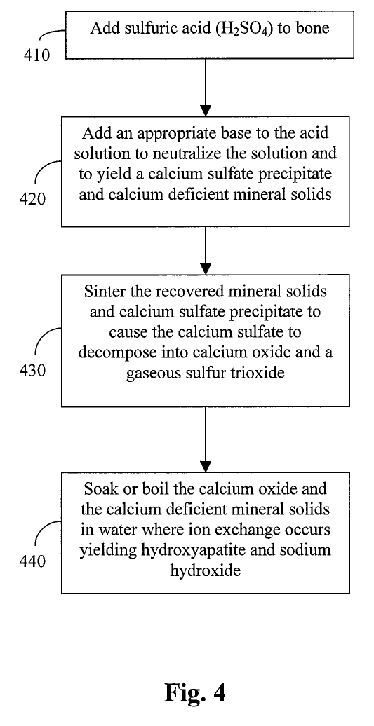

In some methods, insoluble calcium-containing precipitates remaining in solution may be acceptable or desirable, and hydroxyapatite recovery methods may be chosen to result in recovered hydroxyapatite having insoluble calcium-containing precipitates that are not phosphate salts. In such methods, precipitates may be removed and the hydroxyapatite recovered by further processing, as desired. For example, FIG. 4 depicts a mineral recovery method that may yield a calcium precipitate during the process. According to FIG. 4, an acid solution of sulfuric acid (H₂SO₄) may be added to bone 410, and an appropriate base may be added 420 to neutralize the solution to yield calcium-deficient mineral solids and a calcium precipitate in the form of calcium sulfate (CaSO₄). The recovered mineral and calcium sulfate may be sintered 430 so that the calcium sulfate decomposes into calcium oxide and gaseous sulfur trioxide where the gaseous oxide escapes. The calcium oxide may convert to calcium hydroxide from moisture in the air either during the sintering process or while cooling after sintering. To increase conversion of the calcium oxide to calcium hydroxide, the resulting calcium oxide containing mineral solids may be soaked or boiled 440 in water.

Alternatively, the calcium-containing precipitate calcium carbonate (CaCO₃) may result when sodium carbonate is used to neutralize a suitable acid solution, e.g., a mineral acid, to yield the precipitate. When the recovered mineral is heated to a high temperature, e.g., during sintering, the calcium carbonate may decompose to form solid calcium oxide with the carbon leaving as gaseous oxide, such as CO₂ for example. Upon subsequent soaking or boiling in water, or upon using a hydrothermal process, ion exchange may occur such that the sodium incorporated in the hydroxyapatite may be replaced by calcium from the solid calcium oxide, allowing recovery of hydroxyapatite derived from bone.

Multivalent Bases:

Bases of multivalent materials, including but not limited to metals such as Ca, Fe, and Zn, also may be used in the acid neutralization portion of the hydroxyapatite recovery process in accordance with various embodiments. Treatment with a multivalent metal base may result in the multivalent metals being incorporated into the recovered hydroxyapatite. This may be useful in certain applications where modification of hydroxyapatite is desired. For example, a calcium base may be used in excess in the hydroxyapatite recovery process in order to yield tricalcium phosphate (TCP) with or without additional hydroxyapatite.

Treatment with Phosphoric Acid:

According to certain embodiments, treatment with phosphoric acid (H₃PO₄) may be carried out in the hydroxyapatite recovery process, resulting in an acid solution that contains phosphoric acid and monocalcium phosphate (Ca(H₂PO₄)₂). In order to recover hydroxyapatite (i.e., Ca₁₀(PO₄)₆(OH)₂) from this solution, a calcium base such as calcium hydroxide (Ca(OH)₂) or calcium oxide (CaO) may be used to neutralize the acid such that calcium salts are formed from the phosphoric acid neutralization. It will be understood by those skilled in the art that, while calcium bases such as calcium hydroxide and calcium oxide are specifically discussed, other bases, and other calcium bases, may also be used in the neutralization step of the HA recovery process to yield hydroxyapatite derived from bone.

When a calcium base is used to neutralize phosphoric acid in the acid bath, the resulting calcium salts may not be soluble. Therefore, the ratio of calcium to phosphorous may be controlled to yield the desired calcium phosphate compound(s). This is in contrast to using a non-calcium-containing monovalent base for neutralization, in which the amount of added base may not be important because any salts are soluble and capable of being rinsed out.

One effect of using a calcium base for phosphoric acid neutralization is that the amount of calcium base added may increase the amount of calcium in the final material. Therefore, in some embodiments, the Ca:P ratio and phosphate concentration of the acid solution before the neutralization process is determined in order to calculate an amount of calcium base to be added. Alternatively (or additionally), phosphoric acid may be added to the final material to adjust the Ca:P ratio of the final material and mitigate any effects of excess calcium base during neutralization. Thus, in accordance with one embodiment, an excess of calcium base is added, precipitate is recovered and dried, the weight and Ca:P ratio of the dry precipitate are determined, and phosphoric acid is added to adjust the Ca:P ratio.

As previously discussed, in cases where the calcium-containing residues result from the phosphoric acid neutralization process, e.g., calcium carbonate or calcium sulfate, the residues may decompose upon sintering to form calcium oxide, and upon exposure to water (either in liquid or vapor form), the oxide may convert to the hydroxide form, thus resulting in hydroxyapatite.

Organic Acids:

Organic acids such as acetic acid, as well as mineral acids, may be employed in the acid treatment portion of the hydroxyapatite recovery process. Organic acid salts, upon neutralization, may retain calcium. Where calcium is retained by the acid salts, and effectively leached from the recovered hydroxyapatite, the resulting hydroxyapatite may be lower in calcium, or calcium deficient. If the organic acid residues precipitate with the hydroxyapatite upon neutralization, then sintering the hydroxyapatite may burn off the organic portion, resulting in the formation of calcium oxide, and hydroxyapatite may be recovered by the previously mentioned procedure of soaking or boiling the hydroxyapatite in water, or via another hydrothermal process. If, however, the acid/calcium complexes are soluble and are washed out, then extra calcium may be added to the recovered hydroxyapatite.

Chemical Modifications:

In accordance with some embodiments, the recovered bone minerals, such as hydroxyapatite, may be supplemented, further treated or chemically modified.

Inclusion of Tricalcium Phosphate:

Recovered hydroxyapatite from bone may be reacted with a calcium-containing base in order to produce tricalcium phosphate (TCP), i.e., Ca₃(PO₄)₂, which has desirable properties as it is resorbed by the body as it converts to hydroxyapatite over time. Thus, according to some embodiments, natural, bone-derived hydroxyapatite may be processed such that it contains TCP or may be processed to be pure TCP. The amount of TCP resulting from hydroxyapatite reacting with a calcium-containing base depends on the amount of calcium-containing base used. Such processing may comprise treating the recovered hydroxyapatite with phosphoric acid or a less than filly neutralized calcium phosphate to yield TCP. TCP may be unstable in water. Thus, including TCP with the hydroxyapatite product may be useful, for example, in sintered forms of hydroxyapatite, because the TCP transforms into hydroxyapatite over time, which creates an environment for facilitating cell remodeling and resorption.

Addition of Trace Minerals:

In some embodiments, trace minerals may be added as a supplement to recovered hydroxyapatite to achieve desirable biological or physical properties in the final hydroxyapatite material. For example, magnesium or silicon may be added in the form of phosphate salts followed by the appropriate adjustments to control hydroxyapatite and/or TCP content. In another example, trace minerals may be added as an oxide or hydroxide followed by an appropriate addition of phosphoric acid or a less than fully neutralized calcium phosphate to adjust the hydroxyapatite and/or TCP content. Minerals also may be added in any other suitable method.

Addition of Other Additives:

In accordance with some embodiments, other additives, including but not limited to those described below, may be added as a supplement to recovered bone minerals. It will be appreciated that the amount of additive used will vary depending upon the type of additive, the specific activity of the particular additive preparation employed, and the intended use of the composition. The desired amount is readily determinable by one skilled in the art. Any of a variety of medically and/or surgically useful optional substances can be added, or associated with, the recovered hydroxyapatite either before, during, or after recovery or preparation of the hydroxyapatite.

Angiogenesis Promoting Materials

Angiogenesis may be an important contributing factor for the replacement of new bone and cartilage tissues. In certain embodiments, angiogenesis is promoted so that blood vessels are formed at an implant site to allow efficient transport of oxygen and other nutrients and growth factors to the developing bone or cartilage tissue. Thus, angiogenesis promoting factors may be added to the bone minerals to increase angiogenesis. For example, class 3 semaphorins, e.g., SEMA3, controls vascular morphogenesis by inhibiting integrin function in the vascular system, Serini et al., Nature, (July 2003) 424:391-397, and may be included in the recovered hydroxyapatite.

Bioactive Agents

In accordance with some embodiments, recovered bone minerals, such as hydroxyapatite, may be supplemented, further treated, or chemically modified with one or more bioactive agents or bioactive compounds. Bioactive agent or bioactive compound, as used herein, refers to a compound or entity that alters, inhibits, activates, or otherwise affects biological or chemical events. For example, bioactive agents may include, but are not limited to, osteogenic or chondrogenic proteins or peptides; demineralized bone powder as described in U.S. Pat. No. 5,073,373; collagen, insoluble collagen derivatives, etc., and soluble solids and/or liquids dissolved therein; anti-AIDS substances; anti-cancer substances; antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamycin, etc.; immunosuppressants; anti-viral substances such as substances effective against hepatitis; enzyme inhibitors; hormones; neurotoxins; opioids; hypnotics; anti-histamines; lubricants; tranquilizers; anti-convulsants; muscle relaxants and anti-Parkinson substances; anti-spasmodics and muscle contractants including channel blockers; miotics and anti-cholinergics; anti-glaucoma compounds; anti-parasite and/or anti-protozoal compounds; modulators of cell-extracellular matrix interactions including cell growth inhibitors and antiadhesion molecules; vasodilating agents; inhibitors of DNA, RNA, or protein synthesis; anti-hypertensives; analgesics; anti-pyretics; steroidal and non-steroidal anti-inflammatory agents; anti-angiogenic factors; angiogenic factors and polymeric carriers containing such factors; anti-secretory factors; anticoagulants and/or antithrombotic agents; local anesthetics; ophthalmics; prostaglandins; anti-depressants; anti-psychotic substances; anti-emetics; imaging agents; biocidal/biostatic sugars such as dextran, glucose, etc.; amino acids; peptides; vitamins; inorganic elements; co-factors for protein synthesis; endocrine tissue or tissue fragments; synthesizers; enzymes such as alkaline phosphatase, collagenase, peptidases, oxidases, etc.; polymer cell scaffolds with parenchymal cells; collagen lattices; antigenic agents; cytoskeletal agents; cartilage fragments; living cells such as chondrocytes, bone marrow cells, mesenchymal stem cells; natural extracts; genetically engineered living cells or otherwise modified living cells; expanded or cultured cells; DNA delivered by plasmid, viral vectors, or other means; tissue transplants; autogenous tissues such as blood, serum, soft tissue, bone marrow, etc.; bioadhesives; bone morphogenic proteins (BMPs); osteoinductive factor (IFO); fibronectin (FN); endothelial cell growth factor (ECGF); vascular endothelial growth factor (VEGF); cementum attachment extracts (CAE); ketanserin; human growth hormone (HGH); animal growth hormones; epidermal growth factor (EGF); interleukins, e.g., interleukin-1 (IL-1), interleukin-2 (IL-2); human alpha thrombin; transforming growth factor (TGF-beta); insulin-like growth factors (IGF-1, IGF-2); parathyroid hormone (PTH); platelet derived growth factors (PDGF); fibroblast growth factors (FGF, BFGF, etc.); periodontal ligament chemotactic factor (PDLGF); enamel matrix proteins; growth and differentiation factors (GDF); hedgehog family of proteins; protein receptor molecules; small peptides derived from growth factors above; bone promoters; cytokines; somatotropin; bone digesters; antitumor agents; cellular attractants and attachment agents; immuno-suppressants; permeation enhancers, e.g., fatty acid esters such as laureate, myristate and stearate monoesters of polyethylene glycol, enamine derivatives, alpha-keto aldehydes, etc.; and nucleic acids.

In certain embodiments, the bioactive agent may be a drug. In some embodiments, the bioactive agent may be a growth factor, cytokine, extracellular matrix molecule, or a fragment or derivative thereof, for example, a cell attachment sequence such as RGD. A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996; and the United States Pharmacopeia-25/National Formulary-20, published by the United States Pharmcopeial Convention, Inc., Rockville Md., 2001.

In some embodiments, the agent to be delivered may be adsorbed to or otherwise associated with the recovered bone minerals such as hydroxyapatite. The agent may be associated with the hydroxyapatite through specific or non-specific interactions; or covalent or non-covalent interactions. Examples of specific interactions include those between a ligand and a receptor, an epitope and an antibody, etc. Examples of non-specific interactions include hydrophobic interactions, electrostatic interactions, magnetic interactions, dipole interactions, van der Waals interactions, hydrogen bonding, etc. In certain embodiments, the agent may be attached to the hydroxyapatite using a linker so that the agent is free to associate with its receptor or site of action in vivo. In certain embodiments, the agent to be delivered may be attached to a chemical compound such as a peptide that is recognized by the hydroxyapatite. In another embodiment, the agent to be delivered may be attached to an antibody, or fragment thereof, that recognizes an epitope found within the hydroxyapatite. In certain embodiments, at least two bioactive agents may be attached to the hydroxyapatite. In other embodiments, at least three bioactive agents may be attached to the hydroxyapatite. In one embodiment, the inducing agent may be genetically engineered to comprise an amino acid sequence which promotes the binding of the inducing agent to the hydroxyapatite. Sebald et al., PCT/EP00/00637, describes the production of exemplary engineered growth factors suitable for use with the recovered bone minerals.

Hydroxyapatite Products:

Hydroxyapatite recovered from bone using various recovery processes including those described above, or permutations thereof, may be further processed to result in a variety of products containing bone-derived hydroxyapatite having its natural bone mineral inorganic moieties. The implementations described below are an exemplary showing of a variety of hydroxyapatite products or products containing hydroxyapatite derived from bone.

Dry Hydroxyapatite:

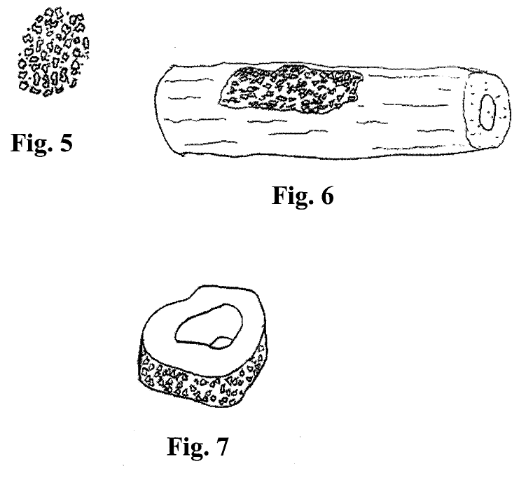

In accordance with some embodiments, the hydroxyapatite may be recovered in a dry form. For example, recovered hydroxyapatite may be spray dried from the slurry, lyophilized, vacuum dried, or dried in a conventional or vacuum oven, e.g., at 200° C. or less, and crushed into a granulate (FIG. 5) or powder form for use as a non-structural bone void filler or a grafting material. In some instances, dry granulate hydroxyapatite may fragment into a fine powder as the recovered hydroxyapatite rehydrates. Thus, according to further embodiments, protein residues may be recovered along with the hydroxyapatite that may cause the granules to be bound together, thereby providing the bound hydroxyapatite with resistance to fragmentation upon hydration. In addition, a binder (such as a biocompatible binder) may be incorporated into the recovered hydroxyapatite, for example, before drying. The binder alternatively may be added in the final wash solution. Regardless of when added, the binder may comprise biocompatible synthetic chemicals such as polyvinyl alcohol (PVA) or carboxymethyl cellulose (CMC), biologically derived materials such as collagen, other binder material, etc. It will be understood by those of skill in the art that any biocompatible material may be used as a binder for recovered hydroxyapatite. One way to incorporate a binder into the hydroxyapatite is to precipitate out soluble proteins from the bone that may be in solution with the recovered hydroxyapatite. Adding a protein precipitating agent (for example, alcohol) to the neutralized hydroxyapatite suspension may cause the protein to be recovered with the hydroxyapatite during the filtration, centrifugation, or settling process. This protein can then act as a binder, such as where the protein has been heat set by, for example, a dehydrothermal polymerization process. Chemical additive-based collagen cross-linking processes can be used in addition or as an alternative to dehydrothermal cross-linking. This process can be enhanced by increasing the amount of protein that is dissolved with the mineral, which may increase the amount of protein potentially available as a binder. For example, the initial goal of the demineralization process is usually to recover the proteins (in the form of DBM) free of mineral. One way of increasing the protein content in the acid-mineral solution is to add bone fines to the recovered acid, and leave the protein from the fines in the solution. Alternatively, the initial demineralization can be performed with the protein (DBM) left with the acid, without recovering the DBM. The solubility of the protein may be enhanced by heating the acid solution before adding base to raise the pH, or by heating the solution after adding base, or by heating both solutions. If the base treated solution is heated, it is advantageous to let it cool before adding alcohol to precipitate the solubilized proteins.

Alternatively, the dried granules or powder may be incorporated into a binder/carrier such as a biocompatible starch, polymer, or flowable material such as glycerol, or other polyalkylene glycols, etc.

Wet Hydroxyapatite:

According to some embodiments, hydroxyapatite may be recovered in a wet form and used as a slurry for implantation (FIG. 6), e.g., as a bone void filler. The hydroxyapatite slurry may be thickened with various biocompatible thickening agents such as starch, PVA, CMC, or other thickening agents in order to form a hydroxyapatite suspension, or paste, for example. Alternatively, the hydroxyapatite may be recovered in a wet form, dried, and rewetted using a sterile saline solution or other suitable solutions or wetting agents.

Ceramic Forms:

In another embodiment, recovered hydroxyapatite may be sintered into a ceramic form. According to some methods, prior to sintering, any standard ceramic processing technique, such as slip casting, molding, or pore forming processes, may be applied. Likewise, after sintering, any standard post sintering ceramic processing technique, such as machining, crushing, and grinding, may be applied in order to achieve the desired final product form.

According to some embodiments, the ceramic forms of hydroxyapatite, e.g., crushed, ground granulates and powders, may be incorporated into any of the binder/carrier systems described above.

In addition, according to some embodiments, the recovered hydroxyapatite in its natural, fortified, or ceramic form may be added to medical implants, for example to skeletal implants (FIG. 7). In certain embodiments, hydroxyapatite may be formed into an osteogenic implant or other medical implant in the shape of a cylinder, square, rectangle, wedge, truncated wedge, cone, conic section, sheet or fiber. Moreover, medical implants formed of hydroxyapatite may also include additives, including but not limited to demineralized bone, mineralized bone, biodegradable polymer, non-biodegradable polymer, collagen, cells, etc. In some embodiments, the implant is configured as a monolithic porous implant with any suitable or desired porosity, including any desired percentage that is greater than 0 and less than 100, between about 1 and about 75%, etc.

The embodiments described above allow hydroxyapatite material to be recovered, resulting in a product that more closely resembles natural bone minerals than other mineral-based bone grafting materials or bone void fillers. Specifically, in many embodiments, natural hydroxyapatite is recovered during demineralization of bone and, thus, during a process that commonly treats hydroxyapatite as waste.

The above-mentioned embodiments are not meant to be limiting. Additional embodiments of the present invention are possible. For example, bone-derived mineral-containing products may be formed into solid or porous blocks, alone or in combination with other biocompatible products. In another example, bone-derived mineral-containing products may be combined with proteins and saline in order to promote bone growth upon implantation. In another embodiment, bone-derived mineral-containing products may be processed by boiling, stirring, and mixing with a proper medium to form a gel-like suspension of bone-derived mineral-containing products. Alternatively, bone-derived mineral-containing products may be mixed with a proper medium to form a putty-like bone-derived mineral-containing material.

Various modifications may be made to the embodiments disclosed herein. For example, bone-derived mineral-containing products may be mixed with synthetic hydroxyapatite or TCP in order to achieve a desired physical property or performance. In addition, bone-derived mineral-containing products may be melted and shaped for an appropriate implantation use.

The above description should not be construed as limiting, but merely as exemplifications of, preferred embodiments. Those skilled in the art will envision other modifications within the scope and spirit of the present disclosure.

What is claimed is:

1. A method for recovering minerals from bone comprising:
separating bone minerals from bone; and
isolating the separated bone minerals, such that the bone minerals are isolated with natural bone mineral inorganic moieties.

2. The method of claim 1, wherein separating comprises treating the bone with an acid extraction solution and a base to neutralize the acid extraction solution.

3. The method of claim 2, wherein the acid and base are selected to yield insoluble bone minerals.

4. The method of claim 1, further comprising drying and crushing the separated bone minerals to form a granulate.

5. The method of claim 4, further comprising adding a binder to the granulate and processing the granulate to yield a paste.

6. The method of claim 1, further comprising spray drying the separated bone minerals to form a granulate.

7. The method of claim 6, wherein the binder comprises bone proteins treated with a chemical additive that causes collagen cross-linking.

8. The method of claim 1, wherein separating bone proteins from bone minerals comprises demineralizing bone, and wherein demineralizing yields insoluble bone-derived hydroxyapatite.

9. The method of claim 8, wherein the bone-derived hydroxyapatite is processed to form tricalcium phosphate.

10. The method of claim 8, wherein the bone-derived hydroxyapatite is processed to form a mixture of tricalcium phosphate and hydroxyapatite.

11. The method of claim 8, wherein the bone-derived hydroxyapatite is processed to incorporate additional minerals

12. The method of claim 11, wherein the additional minerals comprise zinc.

13. The method of claim 11, wherein the additional minerals comprise magnesium.

14. The method of claim 11, wherein the additional minerals comprise calcium.

15. The method of claim 8, wherein the bone-derived hydroxyapatite is processed to incorporate additional supplements.

16. The method of claim 15, wherein the additional supplements comprise at least one angiogenesis promoting material.

17. The method of claim 15, wherein the additional supplements comprise at least one bioactive agent.

18. The method of claim 8, wherein the bone-derived hydroxyapatite is recovered as a wet hydroxyapatite.

19. The method of claim 8, further comprising drying and grinding the isolated bone-derived hydroxyapatite to form a granulate hydroxyapatite.

20. A method for recovering minerals from bone comprising:
acid treating bone in an acid solution to demineralize the bone, such treating with acid resulting in bone minerals having natural bone mineral inorganic moieties;
separating the bone minerals from the acid solution; and
collecting the separated bone minerals.

21. The method of claim 20, wherein separating comprises neutralizing the acid solution by adding a base to the acid solution.

22. The method of claim 21, wherein the solution is neutralized to a pH of at least about 13.

23. The method of claim 20, further comprising treating the separated bone minerals to achieve a neutral pH in the range of about 6 to about 8.

24. The method of claim 23, wherein treatment to achieve a neutral pH comprises washing the separated bone minerals with water.

25. The method of claim 20, further comprising recovering bone-derived proteins with the collected bone minerals, said bone-derived proteins binding the bone minerals to reduce bone mineral fragmentation.

26. The method of claim 25, further comprising adding a chemical additive to cause collagen in the bone-derived proteins to cross-link.

27. The method of claim 25, wherein the protein is precipitated by a protein precipitation agent.

28. The method of claim 20, further comprising adding a binder to the collected bone minerals, said binder binding the bone minerals to reduce bone mineral fragmentation.

29. The method of claim 20, further comprising sintering the collected bone minerals.

30. The method of claim 29, further comprising adding a binder to the sintered bone minerals.

31. The method of claim 20, further comprising adding a pore forming agent to the collected bone minerals and sintering the collected bone minerals with the pore forming agent.

32. The method of claim 20, wherein acid treating bone yields bone minerals that are an insoluble bone-derived hydroxyapatite.

33. The method of claim 32, further comprising processing the bone-derived hydroxyapatite to yield tricalcium phosphate.

34. The method of claim 32, further comprising processing the bone-derived hydroxyapatite to yield a mixture of hydroxyapatite and tricalcium phosphate.

35. The method of claim 32, further comprising processing the bone-derived hydroxyapatite to incorporate additional minerals into the hydroxyapatite.

36. The method of claim 32, further comprising processing the bone-derived hydroxyapatite to incorporate additional supplements into the hydroxyapatite.

37. The method of claim 36, wherein the additional supplements comprise at least one bioactive compound.

38. The method of claim 32, wherein the bone-derived hydroxyapatite is recovered as a wet hydroxyapatite.

39. The method of claim 25, further comprising drying and grinding the bone-derived hydroxyapatite to form a granulate.

40. A method for recovering minerals from bone comprising:
acid treating bone in an acid solution to demineralize the bone, such treating with acid resulting in hydroxyapatite having natural bone mineral inorganic moieties;
separating the hydroxyapatite from the acid solution; and
collecting the separated hydroxyapatite.

41. An implantable hydroxyapatite material, wherein at least a portion of the implantable hydroxyapatite material is separated from at least partially dissolved bone and comprises natural bone mineral inorganic moieties.

42. The implantable hydroxyapatite material of claim 41, wherein the hydroxyapatite material is a granulate.

43. The implantable hydroxyapatite material of claim 41, wherein the hydroxyapatite material is a paste.

44. The implantable hydroxyapatite material of claim 41, wherein the hydroxyapatite material is a mixture of hydroxyapatite and bone protein.

45. The implantable hydroxyapatite material of claim 41, wherein the hydroxyapatite material is a mixture of hydroxyapatite and a binder.

46. The implantable hydroxyapatite material of claim 41, wherein a portion of the hydroxyapatite material is a hydroxyapatite-derived tricalcium phosphate.

47. The implantable hydroxyapatite material of claim 46, wherein the hydroxyapatite and the hydroxyapatite-derived tricalcium phosphate comprise sintered hydroxyapatite and hydroxyapatite-derived tricalcium phosphate.

48. The implantable hydroxyapatite material of claim 46, wherein the sintered hydroxyapatite and hydroxyapatite-derived tricalcium phosphate further comprises a binder.

49. The implantable hydroxyapatite material of claim 41, wherein the hydroxyapatite material is a supplemented hydroxyapatite material.

50. The implantable hydroxyapatite material of claim 49, wherein the supplemented hydroxyapatite material is a trace mineral-supplemented hydroxyapatite.

51. The implantable hydroxyapatite material of claim 41, wherein the hydroxyapatite material is a bone void filler.

52. The implantable hydroxyapatite material of claim 41, wherein the hydroxyapatite material is a bone grafting material.

53. The implantable hydroxyapatite material of claim 41, wherein the hydroxyapatite material is incorporated onto a medical implant.

54. The implantable hydroxyapatite material of claim 41, wherein the implantable hydroxyapatite material is processed to incorporate additional additives.

55. The implantable hydroxyapatite material of claim 54, wherein the additional additives comprise at least one angiogenesis promoting factor.

56. The implantable hydroxyapatite material of claim 54, wherein the additional additives comprise at least one bioactive agent.

57. An osteogenic implant made from the material of claim 41.

58. The implant of claim 57, wherein the implant is configured as a monolithic implant with a porosity between about 1 and about 75%.

59. The implant of claim 57, wherein the implant is configured as a cylinder, square, rectangle, wedge, truncated wedge, cone, conic section, sheet, or fiber.

60. The implant of claim 57, further comprising one or more of demineralized bone, mineralized bone, biodegradable polymer, non-biodegradable polymer, collagen, and cells.
